SEQUENCE LISTING (7 sequences)

SEQ ID NO: 1 - protein, 4 aa, Artificial Sequence
  Description of Artificial Sequence Synthetic peptide
APGG

SEQ ID NO: 2 - protein, 4 aa, Artificial Sequence
  Description of Artificial Sequence Synthetic peptide
APGW

SEQ ID NO: 3 - protein, 4 aa, Artificial Sequence
  Description of Artificial Sequence Synthetic peptide
NMWG

SEQ ID NO: 4 - protein, 4 aa, Artificial Sequence
  Description of Artificial Sequence Synthetic peptide
NMWW

SEQ ID NO: 5 - protein, 9 aa, Artificial Sequence
  Description of Artificial Sequence Synthetic peptide
APWNMGNMW

SEQ ID NO: 6 - protein, 12 aa, Artificial Sequence
  Description of Artificial Sequence Synthetic peptide
APGWNMWWNMWG

SEQ ID NO: 7 - protein, 15 aa, Artificial Sequence
  Description of Artificial Sequence Synthetic peptide
APWAPGWNMWWNMWG

(19) United States
(12) Patent Application Publication
(10) Pub. No.: US 2021/0020270 A1
(43) Pub. Date: Jan. 21, 2021

(54) CONSTRAINED DE NOVO SEQUENCING OF NEO-EPITOPE PEPTIDES USING TANDEM MASS SPECTROMETRY

(71) Applicant: THE TRUSTEES OF INDIANA UNIVERSITY, Bloomington, IN (US)

(72) Inventors: Haixu TANG, Bloomington, IN (US); Sujun LI, Bloomington, IN (US); Alex DECOURCY, Princeton, NJ (US)

(21) Appl. No.: 16/978,390

(22) PCT Filed: Mar. 8, 2019
(86) PCT No.: PCT/US2019/021306

§ 371 Date: Sep. 4, 2020

Related U.S. Application Data
(60) Provisional application No. 62/640,319, filed on Mar. 8, 2018.

(51) Int. Cl. G16B 40/10; G01N 33/68

(52) U.S. Cl. CPC G16B 40/10; G01N 2560/00; G01N 33/6848

(57) ABSTRACT

Technologies for identifying one or more neoepitope peptides include generating a database that includes peptide sequences within a predefined range of length of residues, assigning a prior probability to each of the peptide sequences in the database, obtaining mass spectra of a plurality of fragments of a target molecule produced by mass spectrometry, determining, for each mass spectrum, matching scores of peptide-spectrum matches between the mass spectra and the peptide sequences in the database as a function of prior probabilities of the peptide sequences and matching probabilities, and determining a subset of the peptide-spectrum matches that has a corresponding matching score higher than a threshold.

### TECHNICAL FIELD

The present disclosure relates generally to methods for protein analysis involving mass spectrometry.

### BACKGROUND

The peptide epitopes presented by major histocompatibility complex class I (MHC-I) molecules on cell surfaces display a representative image of the collection of (endogenously synthesized or exogenous) proteins in the cell, allowing immune cells (e.g., the CD8⁺ cytotoxic T-cells) to monitor the biological activities occurring inside the cell, a process known as the immune surveillance. A typical process of the peptide processing and presentation involves three steps: 1) the cytosolic proteins are first degraded into peptides by the proteasome; 2) the resulting peptides are loaded onto MHC-I molecules; and 3) the MHC-I/peptide complex is transported into the plasma membrane of the cell via endoplasmic reticulum (ER), while the extracellular domain of MHC-I, where the epitope peptide binds, is exported outside the membrane. In normal cells, the peptides presented by MHC-I will not induce immune responses. However, when abnormal processes (e.g., viral infection or tumorigenesis) occur inside cells, a fraction of MHC-I molecules may present peptides from foreign or novel proteins (e.g., due to somatic mutations in tumor cells), often referred to as the neoepitope peptides or neoantigens. Consequently, the cells presenting such peptides will likely to be recognized and subsequently killed by cytotoxic T-cells.

During tumor development, maintenance and progression, tumor cells accumulate thousands of somatic mutations, many of these occurring in protein-coding regions of tumor genes. Among them, missense or frameshift mutations have the potential to generate neoepitope peptides, which can be used as biomarkers for characterizing the states and subtypes of cancer, or can be selected as potential therapeutic cancer vaccines to induce robust and tumor-specific responses. Furthermore, neoepitope peptides were recently demonstrated as potential targets in cancer immunotherapies such as adoptive T-cell therapy.

### BRIEF DESCRIPTION OF THE DRAWINGS

The detailed description particularly refers to the following figures, in which:

FIG. 1 is an example of positional specific scoring matrix (PSSM) (shown as a frequency heatmap) derived from neoepitope peptides of 9 amino acid residues bound to HLA-C*0501, the third position is dominated by Asp while at the ninth position, Leu and Val are preferred;

FIG. 2 is a schematic illustration of the exploration of the peptide sequence space in the constrained de novo algorithm;

FIG. 3 is score distributions of peptide-spectrum matches (PSMs) reported by the constrained de novo sequencing algorithm and the decoy PSMs from the reverse peptides;

FIG. 4A illustrates length distributions of the top-ranked peptides reported by the constrained de novo sequencing algorithm;

FIG. 4B illustrates the sequence logos representing the position specific frequency pattern among the top-ranked peptides with different lengths;

FIG. 5A illustrates the comparison of PSMs and identified unique peptides (in parentheses) reported by database searching and constrained de novo sequencing;

FIG. 5B illustrates a number of amino acids difference in overlapped IDs from database search and constrained de novo;

FIG. 5C illustrates the prior probability and matching scores of the PSMs reported by the constrained de novo sequencing and database search approach. The PSMs are depicted in different colors: orange for those detected by both approaches, red for those detected by database searching only, and black for those detected by de novo sequencing only while blue for those reported by de novo sequencing and also have at least 50% sequence similarity to human proteins; and

FIG. 6 is an example of positional specific scoring matrix (PSSM) (shown as a frequency heatmap) derived from neoepitope peptides of 11 amino acid residues bound to HLA-C*0101.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

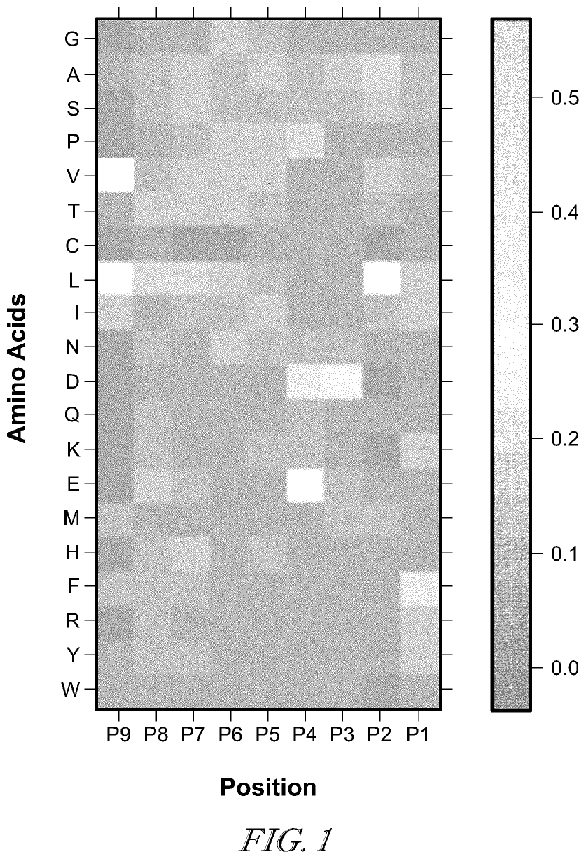

Neoepitope peptides are newly formed antigens presented by major histocompatibility complex class I (MHC-I) on cell surfaces. The cells presenting neoepitope peptides are recognized and subsequently killed by cytotoxic T-cells Immunopeptidomic approaches aim to characterize the peptide repertoire (including neoepitope) associated with the MHC-I molecules on the surface of tumor cells using proteomic technologies, providing critical information for designing effective immunotherapy strategies. In the present application, a constrained de novo sequencing algorithm was developed to identify neoepitope peptides from tandem mass spectra acquired in immunopeptidomic analyses. The constrained de novo sequencing method incorporates prior probabilities to putative peptides according to position specific scoring matrices (PSSMs) representing the sequence preferences recognized by MHC-I molecules, as illustrated in FIG. 1. A dynamic programming algorithm was implemented to determine the peptide sequences with an optimal posterior matching score for each given MS/MS spectrum. Similar to the de novo peptide sequencing, the dynamic programming algorithm allows an efficient searching in the entire peptide sequence space. On an liquid chromatography coupled tandem mass spectrometry (LC-MS/MS) dataset, the performance of the constrained de novo sequencing algorithm in detecting the neoepitope peptides bound by the Human Leukocyte Antigen (HLA)-C*0501 molecules was demonstrated to be superior to database search approaches and existing de novo peptide sequencing algorithms.

In the past decade, clinical evidence has been accumulated on tumor-specific immune activities, leading to the implementation of successful strategies of cancer immunotherapy. Because of the strong implications of neoepitope peptides in the design of effective cancer immunotherapy, different genomic and proteomic methods have been developed to identify neoepitope peptides presented by tumor cells from cancer patients. The genomic approaches start from exon and transcriptome sequencing of normal and tumor tissues in attempt to identify proteins over- or under-expressed tumor issues, as well as missense or frameshift mutations in tumor proteins, and then use computational methods to predict neoepitope candidate from these tumor proteins based on the immunogenicity of peptides, i.e., the likelihood of peptides being presented by MHC-I molecules in tumor cells and furthermore likely to provoke an immune response. Notably, the genomic approaches may not report accurate neoepitope peptides due to various limitations of the methods. First, some very low abundant proteins that may not be identified using transcriptome sequencing are often presented by the MHC-I molecules, and can provoke robust immune responses. Second, current immunogenicity prediction algorithms cannot yet accurately model the process of antigenic peptide processing and presentation by MHC-I, and thus may report many false positives and false negatives of neoepitope peptides. Most importantly, as multiple MHC-I molecules are encoded by the highly polymorphic human leukocyte antigen (HLA) genes (including three major types of HLA-I, HLA-II and HLA-III) in an individual patient, the peptide immunogenicity is indeed a private measure specific to this cancer patient, and thus cannot be modeled without sufficient neoepitope peptides already identified from the patient's own sample.

In contrast, the immunopeptidomic approaches aim to directly analyze the peptide repertoire bound by the MHC-I molecules on the surface of tumor cells using proteomic technologies, and thus can overcome the limitations of genomic approaches. Because of its high throughput and sensitivity, liquid chromatography coupled tandem mass spectrometry (LC-MS/MS) has been used in proteomics in an attempt to identify and quantify proteins in complex protein mixtures, and also used for identifying neoepitope peptides eluted from MHC molecules. From the MS/MS spectra acquired in an immunopeptidomic experiment, potential neoepitope peptides are identified often using a database search engines designed for peptide identification in proteomics (e.g. Sequest, Mascot or MSGF⁺). However, the neoepitope peptides have some distinct features comparing to the peptides from general proteomic analysis. On one hand, neoepitope peptides bound to different classes of MHC-I molecules have relatively fixed length; for example, human HLA class I (HLA-I) recognizes peptides 8 to 12 amino acid residues in length. On the other hand, unlike the peptides in proteomic experiments typically from tryptic digestion at specific basic amino acid residues, neoepitope peptides can be cleaved by proteasome at any arbitrary position in the target proteins. As a result, when MS/MS spectra from an immunopeptidomic study is searched against a target protein database (e.g, consisting of all human proteins), all non-tryptic peptides of the lengths within a range (8-12 residues) are considered; in the human protein database, there are ≈10⁷-10⁸ such peptides, much greater than the number of tryptic peptides (≈10⁶). Furthermore, a large fraction (about a third) of neoepitope peptides are generated by proteasome-catalyzed peptide splicing (PCPS) that cuts and pastes peptide sequences from different proteins. If all concatenate peptides (with two subpeptides from the same or different proteins) are considered in the database search, the number of target peptides increases to ≈10¹⁵, close to the total number of peptides 8-12 residues in length. which poses great challenges to database search not only on the running time but also on potential false positives in peptide identification. Finally, strong sequence patterns are present in neoepitope peptides, largely because of the preferences in the binding affinity and specific structures of MHC-I molecules. The sequence pattern in neoepitope peptides recognized by a specific class of MHC-I molecule can be represented by a positional specific scoring matrices (PSSMs; see FIG. 1 as an example for HLA-C), or more complex machine learning models for predicting peptide immunogenicity. However, these sequence information are not used by current approaches for neoepitope peptide identification in proteomic experiments.

De novo peptide sequencing algorithms (such as Peaks, pepNovo, pepHMM, uniNovo, Novor, and DeepNovo) represent a different approach to peptide identification in proteomics that attempt to reconstruct the peptide sequence directly from an MS/MS spectrum. Comparing to database search algorithms, de novo sequencing algorithms explore the entire space of peptides, but are often more efficient because of the employment of a dynamic programming algorithm. From a Bayesian perspective, the database search approach can be viewed as a special case of de novo peptide sequencing, which assumes that only the proteins in the database can be present in the sample, and thus the peptides from these proteins have the prior probabilities of 1 while the other peptides have the prior probabilities of 0. Previous studies have showed that although the top peptide reported by the de novo sequencing algorithm for an MS/MS spectrum was often incorrect, the correct one was usually the peptide in the database that received the highest score in de novo sequencing, indicating that the incorporation of the protein database as prior knowledge significantly improves peptide identification.

In the present application, a novel constrained de novo sequencing algorithm for neoepitope peptide identification is presented. The constrained de novo sequencing method includes the de novo sequencing and the database searching algorithms. For example, it explores the entire space of peptide sequences 9-12 residues in length but assigns a different prior probability to each putative peptide according to MHC-I specific PSSMs, such that the peptide with a motif with high immunogenicity incorporates a high prior probability into the posterior probability score of the peptide-spectrum matches (PSMs). Utilizing the sequential property of the PSSMs, the dynamic programming (DP) algorithm was extended for de novo peptide sequencing to determine the peptide sequences with the optimal posterior matching scores for each given MS/MS spectrum. Notably, similar to de novo peptide sequencing algorithms, the dynamic programming algorithm allows an efficient searching in the entire peptide sequence space, which, as shown above, is comparable to the size of the database consisting of all putative neoepitope peptides (including the concatenate peptides) derived from human proteins. The constrained de novo sequencing algorithm in a LC-MS/MS dataset was tested for detecting the neoepitope peptides bound by the HLA-C*0501 molecules. The constrained de novo sequencing method could detect about 19,017 neoepitope peptides of lengths between 9 to 12 residues with estimated false discovery rate below 1%. In contrast, the database search approach (using MSGF⁺ against the human protein database) identified about 4,415 PSMs (1,804 unique peptides), in which 2,104 PSMs (764 unique peptides) have the length between 9 to 12 residues as putative neoepitope peptides. Out of the 2,104 PSMs, 1,269 were also identified by the constrained de novo sequencing method. A majority (791 out of 1,269) of the PSMs were exact matches, while most (360 out of 478) remaining PSMs contain only a swap of consecutive residues in peptide sequences. Finally, a conventional de novo sequencing algorithm uniNovo was also tested on the same dataset. It reported sequence tags on 1,863 MS/MS spectra, but with low sequence coverage (on average 3 amino acid residues per peptide), and thus cannot be used in neoepitope peptide sequencing. These results imply that the constrained de novo sequencing algorithm benefit from the prior probabilities (provided by the PSSMs) to distinguish the most likely neoepitope peptides from other peptides sharing similar sequences.

Constrained de novo peptide sequencing. Given an MS/MS spectrum M, the constrained de novo peptide sequencing problem is to find the peptide sequence T within a range of length (Iₘᵢₙ<|T|≤Iₘₐₓ) that maximizes a posterior matching score S:

Score(M,T)=P(T)·P(M|T) (1)

where P(T) represents the prior probability of the peptide T, and P(M|T) represents the matching probability, i.e., the probability of observing the MS/MS spectra from the peptide T. For peptides with a fixed length l, their prior probabilities are defined by a PSSM

$${p}_{i j} ( {\sum }_{i} {p}_{i j} = 1 )$$

for residue i at the position j (j=1, 2, . . . , l) in the peptide; thus, for the peptide

$$T = {t}_{1} {t}_{2} … {t}_{l} , P ( T ) = {\prod }_{j = 1}^{l} {p}_{{t}_{j} j} .$$

The matching probability P(M|T) is modeled by the independent fragmentation at each peptide bond:

$$P ( M \vert T ) = {\prod }_{j}^{l} = 1 P ( {f}_{M , j} ) ,$$

where P(f_{M,j}) stands for the probability of observing f_{M,j}, the occurrence pattern of the set of fragment ions, including the b-ion, y-ion and the neutral loss ions, derived from the fragmentation between the precursor (t₁t₂ . . . tⱼ) and the suffix (tⱼ₊₁ tⱼ₊₂ . . . tₗ) peptide in M. Notably, f_{M,j} is dependent only on mⱼ, the j-th prefix mass of the prefix peptide t₁t₂ . . . tⱼ, but is not dependent on the peptide sequences. Therefore,

$$Score ( M , T ) = {{\prod }_{j = 1}}^{l} [ {p}_{{t}_{j} j} P ( F ( {m}_{j} ) ) ] \qquad ( 2 )$$

where P(F(mⱼ)) represents probability of observing the set of fragment ion F(mⱼ) associated with the prefix mass mⱼ in M. These probabilities can be learned from a training set of identified MS/MS spectra, in which the peaks are assigned. Alternatively, as adopted here, P(F(mⱼ)) is assigned empirically based on the logarithm transformed ion intensities of the matched b- or y-ions (within a mass tolerance). Let S(j,m) be the maximum posterior matching score between an MS/MS spectrum and any peptide of length j with a total mass of m, which can be computed by using a dynamic programming algorithm,

S(j,m)=max_{k∈A}[S(j−1,m−k)·[p_{j,k}·P(F(m))] (3)

where k is amino acid in the alphabet A. Note that the multiplication of probabilities in equation 3 can be transformed into the summation of the logarithms of probabilities. Finally, the optimal potential matching score of a peptide with a fixed length l, implicated as the number of columns in the PSSM, matching a given spectrum M, is S(M;l,mₚᵣ), in which mₚᵣ is the precursor mass of M. The algorithm can be applied to each putative peptide length between lₘᵢₙ and lₘₐₓ with a corresponding PSSM, and the peptides will be reported in the order of their posterior matching scores. The dynamic programming algorithm is executed in O(l·mₚᵣ) time using P(l·mₚᵣ) space (where the fragment ion masses are binned according to the mass resolution), but can be further accelerated by heuristics as described below. It should be appreciated that the prefix mass scoring may be used in de novo peptide sequencing, database searching and spectrum alignment to identify mutations and post-translation modifications (PTMs). The dynamic programming algorithm presented here can be view as matching a predefined PSSM against a vector of prefix mass scores (probabilities) in order to find the optimal matches between a peptide and a subset of prefix masses.

Accelerating the dynamic programming algorithm. For an input MS/MS spectrum of the precursor mass mₚᵣ and a PSSM with a specific neoepitope peptide length l, the above algorithm explores all potential prefix masses between 0 and mₚᵣ for each prefix peptide of the length from 0 to 1. However, there are only a limited number of prefix masses corresponding to prefix peptides of a fixed length, indicating that the matrix of S(j,m) computed in equation 3 has many zeroes, especially when for small j. To compute only the non-zero elements in S(j,m), a branch-and-bound approach was exploited to explore the peptide space, while retaining only the best scored sub-peptide among those with the same prefix mass.

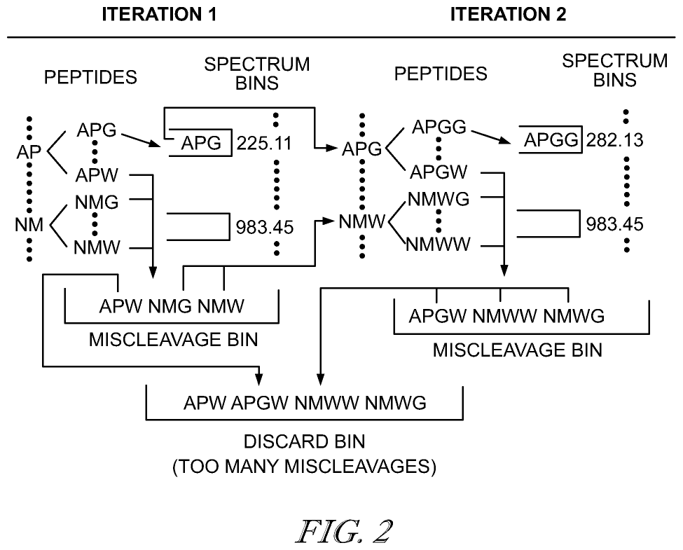

The sequencing algorithm maintains a pool of putative prefix peptides, each associated with a posterior matching score. The pool starts with N (N=|A|=20 representing the number of amino acid masses) prefix peptides of length l (FIG. 2) with posterior matching scores of S(1,m(k))=p₁ₖ:·P(F(m(k))) (where m(k) is the mass of the amino acid k). At each following iteration j, for j=2, . . . , l, every prefix peptide in the pool generates N new prefix peptides, one for every amino acid, by appending a new amino acid to the end of each existing peptide (of length j−1) in the pool.

After appending an amino acid k to an existing prefix peptide with mass m′, the mass of the resulting prefix peptide (i.e., the prefix mass m) is used to compute P(F(m)), and then the posterior matching score of the new prefix peptide is computed by S(j,m)=S(j−1,m′)·pⱼₖ·P(F(m)), where S(j−1,m′) is the posterior matching score associated with the existing prefix peptide of length j−1. At each step, the precursor mass m should match at least one of b- and y-ions; otherwise, the precursor peptide is labeled with one miscleavage, which is tracked on each iteration of an algorithm: if a prefix peptide contains too many miscleavages, it is eliminated from further extension. Once the posterior matching score of a prefix peptide is obtained, it will be compared with other peptides in the pool with the same prefix mass, and only the one with the higher score is retained. After each step, at most N×mₚᵣ prefix peptides are retained in the pool. The algorithm is illustrated in FIG. 2. It should be noted that, although the worst-case running time of the de novo sequencing algorithm is still O(l·mₚᵣ) for each spectrum, in practice, it runs much faster as many un-realistic prefix masses were not evaluated, especially for small l.

In the final step (with prefix peptides of the expected length l), all peptides with masses matching the precursor mass are re-assessed by using a global scoring scheme (see below), and are reported in the order of their global scores. Note that for each input MS/MS spectrum, the constrained de novo algorithm was conducted four times, with an input PSSM for peptides of length 9, 10, 11 and 12, respectively.

Pre-processing of MS/MS spectra. Prior to constrained de novo sequencing algorithm, several pre-processing steps were conducted on the MS/MS spectra, including: 1) peaks with an intensity of 0 were removed; 2) the precursor peak was removed; 3) any converted mass greater than precursor mass was removed; 4) Isotopic masses of precursor masses were removed; 5) the intensities of all peaks were logarithm-transformed.

Construction of PSSMs. Peptides of length 9-12 were extracted from the IEDB database http://www.iedb.org/, and separated by length. A total of 892 peptides of length 9, 191 peptides of length 10, 110 peptides of length 11, and two peptides of length 12 were considered. Four PSSMs were created, one for each peptide length, in which the amino acid frequency in every position in the PSSM was computed based on these peptide sequences and the pseudo-count of 1 was incorporated to ensure there were no frequencies of 0.

Re-assessment of peptide-spectrum matches (PSMs) by global scoring. The global score of a PSM is a probability measure, based on a combination of the prior probability based on the input PSSM, and how well its theoretical fragmentation of the peptide matches to the experimental spectrum. It is calculated using equation (1), where P(T) is the probability of the peptide given the PSSM, normalized to the length of the peptide, and P(M|T) is the probability of observing MS/MS M from peptide T based off of the theoretical fragmentation of T. P(M|T) is calculated by

$$Score ( A , E , W ) = 1 - {\sum }_{i = 1}^{k} \frac{{a}_{i} \cdot {e}_{i}}{W} \qquad ( 4 )$$

where eᵢ is a normalized intensity of the experimental spectrum E, aᵢ is the mass accuracy (in ppm) between experimental mass i and theoretical fragmentation mass i (or W if there is no matching mass between the two), from the mass accuracy vector A, W is the lowest allowable mass accuracy between an experimental and theoretical mass, and k is the number of peaks in the experimental spectrum M.

False discovery rate estimation. After the global scores were computed for all PSMs, it was necessary to determine a score threshold to validate whether a peptide match was reliably identified from an MS/MS spectrum by the constrained de novo sequencing algorithm. Note that it is possible for multiple similar peptide sequences to score high enough to indicate that any of them could be the correctly identified neoepitope peptide producing the corresponding MS/MS spectrum. In this case, the de novo sequencing algorithm reports all of them. As shown in the results section, in practice, usually only a few peptides (2) are reported for each spectrum.

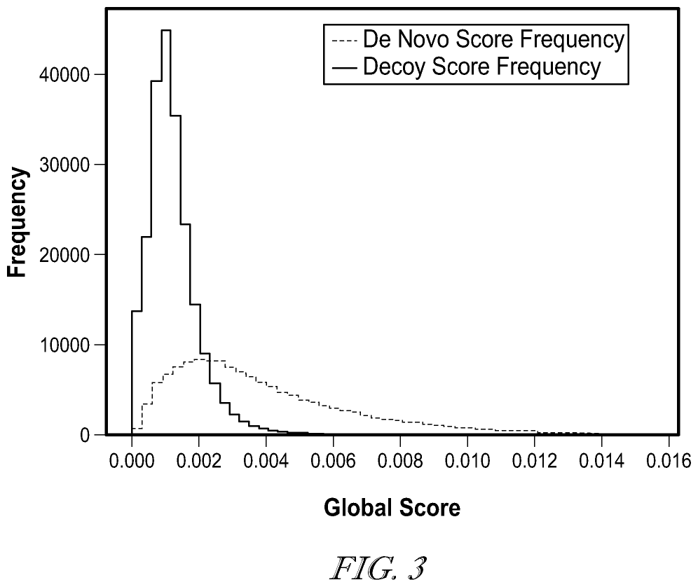

To obtain an appropriate score threshold, similar strategy to the target-decoy search in database searching was adopted to estimate the false discovery rate (FDR) of PSMs. A decoy peptide database consisting of about 40 million randomly selected and reversed peptides with lengths of 9-12 residues from the proteins in the Uniprot database was generated. Additionally, a second database was created for the reversed peptides found by the constrained de novo sequencing algorithm. For each spectrum in the analysis, up to 10 peptides matching the spectrum precursor mass within the mass resolution (35 ppm) were selected from both databases as decoys. The top scoring peptides among these decoy peptides were used to form the decoy PSMs, whose global scores were computed. The score distributions are depicted in FIG. 3, containing the scores from both decoy PSMs and the PSMs reported by the constrained de novo sequencing algorithm. The following formula was used to estimate the FDR at a certain score threshold t: FDRₜ=N_{decoy}/N_{cons}, where N_{decoy} and N_{cons} represent the numbers of decoy and positive (from the sequencing algorithm) PSMs with global scores above t, respectively. The PSMs with the global score higher than 0.0058 was estimated to have FDR lower than 1%.

Datasets. The dataset was obtained from ProteomeXChange (accession number: PXD006455). The experiments were conducted on two common HLA-C: HLA-C*05:01 and HLA-C*07:02. These HLA class I molecules were isolated from the cell surface of C*05 and C*07 transfected 721.221 cells, and sequenced bound peptides by mass spectrometry. HLA-C*05:01 has higher expression level and more diversified binding peptides. In the testing, the binding peptides of HLA-C*05:01 (with length between 9 to 12 residues) was chosen to demonstrate the performance of the constrained de novo sequencing method. In total, there are 339,513 spectra acquired in a total 25 fractions of LC-MS/MS analysis using the Q Exactive HF-X MS (Thermo Fisher Scientific).

Database Searching. MSGF⁺ was used as the database searching engine. The parameters for the MSGF⁺ are set as following to match the experimental conditions of the LC-MS/MS analyses: 1) instrument type: high-resolution LTQ; 2) the enzyme type: unspecific cleavage; 3) precursor mass tolerance: 15 ppm; 4) isotope error range: −1, 2; 5) modifications: oxidation as variable and carboamidomethyl as fixed; 6) maximum charge is 7 and minimum charge is 1. The FDR is estimated by using a target-decoy search approach (TDA).

Results

Constrained de novo sequencing. In the illustrative embodiment, the constrained de novo sequencing algorithm is implemented in C. It spends a total of 8,910 minutes on a Linux computer (Intel® Xeon® CPU ES-2670 0 @ 2.60 GHz) as single thread to process 339,513 input MS/MS spectra in the HLC-C peptidomic dataset, i.e., about 1.6 seconds per MS/MS spectrum. However, it should be appreciated that any coding language may be used to implement the constraint de novo sequencing algorithm and any computing device may be used to execute the de novo sequencing algorithm. In the illustrative embodiment, among the entire set of spectra, the sequencing algorithm reported one or more peptide sequences for 136,249 (40.14%) spectra, resulting a total of 2,775,977 peptide-spectrum matches (PSMs), i.e., 20 PSMs (peptides) per spectra. Among them, 81,888 PSMs over 28,759 spectra (i.e., 2.85 PSMs per spectra) received a global matching score above 0.0058 (corresponding to about 1% FDR; see Methods), corresponding to 57,449 unique peptides, are retained for further analysis.

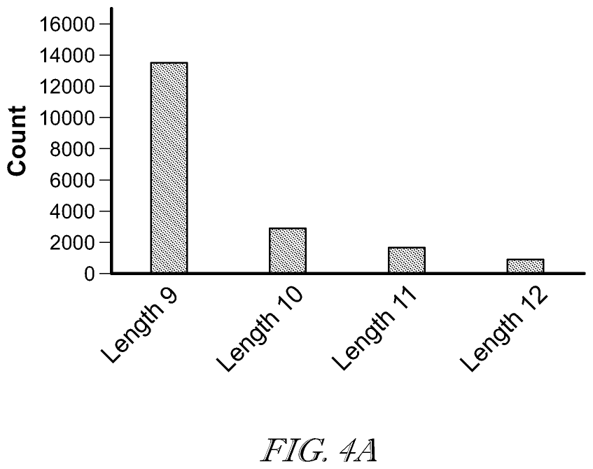

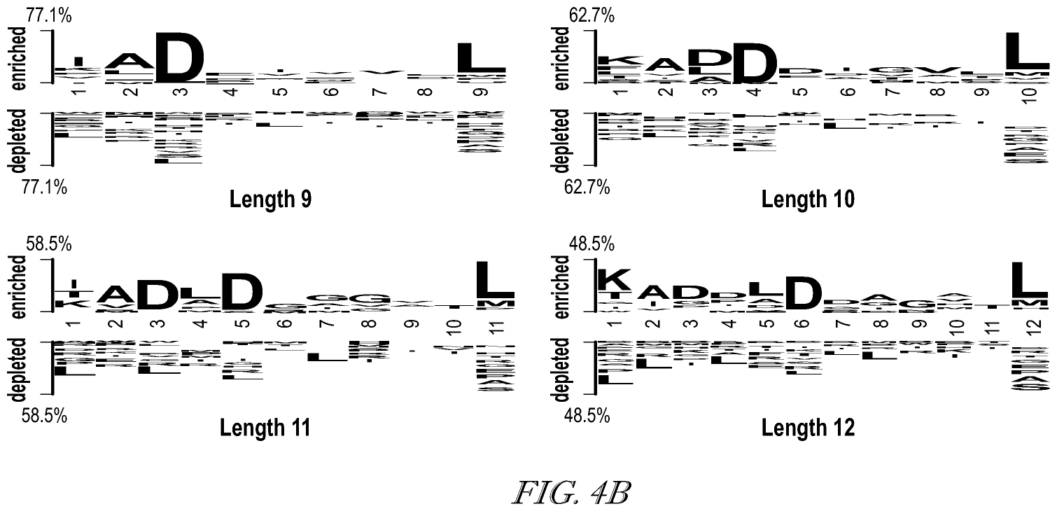

The top-ranked peptides of the 28,759 spectra corresponds to 19,017 unique peptides. The length distribution of these peptides is illustrated in FIG. 4. A majority (13,648, 71.76%) of them are 9 residues in length, which is consistent with previous observations and the IEDB database, in which 892 out of 1,195 (74.64%) HLA-C*0501 bounded peptides are 9 residues in length. FIG. 4B shows the sequence logo generated by using the identified peptides by the de novo sequencing method. Specifically, 13,648 peptides have 9 residues, 2,904 have 10 residues, 1,647 have 11 residues, and 818 have 12 residues. Those sequences were used to generate the sequence logos in FIG. 4. For peptides of length 9, the sequence logo showed that the positions of P2, P3 and P9 have strong amino acid preferences: P2 is enriched by Ala, P9 is enriched by Leu/Ile, and P3 is dominated by Asp. For peptides of other lengths, Asp is predominant at multiple positions, especially in the peptides N-termini, while Leu/Ile are predominant in peptides C-termini.

If all the sequences are retained as long as the global matching score is above the threshold, the constrained de novo sequencing method reported 57,449 unique peptide sequences. All the de novo sequences was kept here, because in many cases multiple peptide sequences containing swapped consecutive amino acids are reported, possibly due to missing fragment peaks to distinguish them in the MS/MS spectra. For those cases, the constrained de novo peptide sequencing algorithm will report very similar peptides with nearly identical global matching scores.

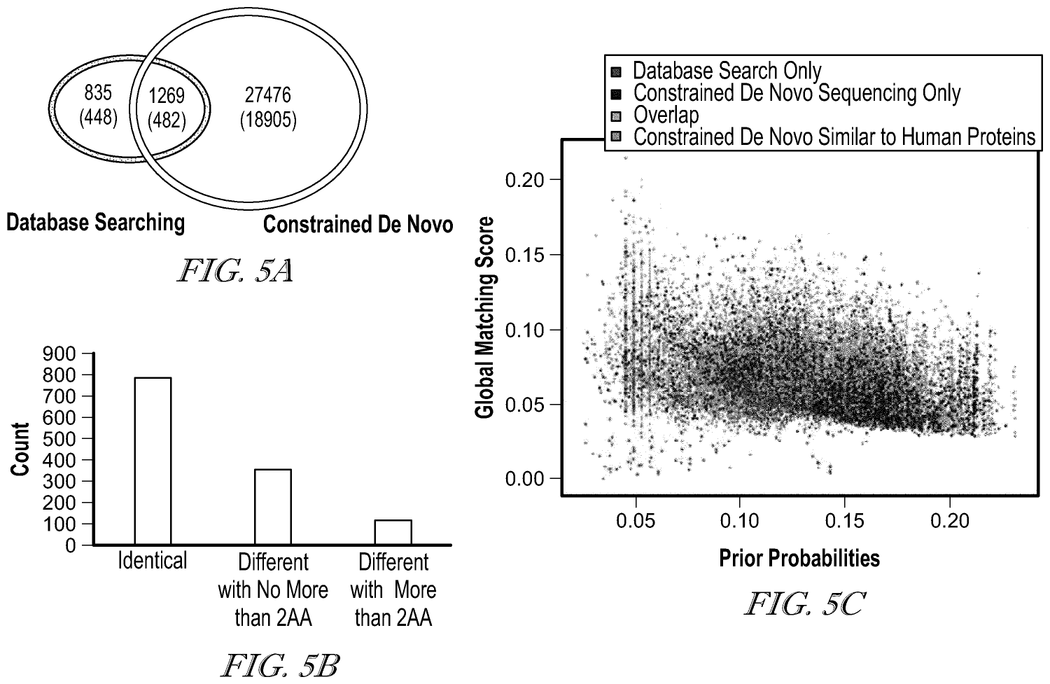

Comparison with Database Searching Results. MSGF⁺ is employed to identify peptides by searching against the human proteome database. The computation takes 1,102 minutes on a Linux computer (Intel® Xeon® CPU ES-2670 0 @ 2.60 GHz). It reported 4,415 PSMs given 5% false discovery rate. It should be appreciated that when a more common FDR threshold 0.01 is used, much fewer (1,280) MS/MS spectra were identified, among which only 97 were identified as peptides with lengths between 9 and 12. Among these PSMs, 2,104 are identified as peptides of lengths between 9 to 12 residues (corresponding to 764 unique peptide sequences), which are putative HLA-C*0501 bounded neoepitope peptides. The peptides identified by the constrained de novo sequencing algorithm were compared with those identified by the database searching method in a Venn diagram shown in FIG. 5A. A total of 1,269 spectra are identified by both the database searching and the de novo sequencing method, among which 791 spectra were identified as identical peptides by both methods: for 360 spectra, the peptides identified by the de novo sequencing method differ only in no more than two amino acid residues from the peptides identified by the database searching (where most of cases are two consecutive residues swaps); and for the remaining 118 spectra, the two identified peptides by these two methods differ in more than two residues, but share over 50% sequence similarity. It should be appreciated that the top-ranked peptides reported by the de novo sequencing algorithm were considered, and ILE and LEU are considered as identical amino acids in this comparison.

The PSMs reported by both the database searching and the de novo sequencing algorithm, and those reported by only one of these methods were investigated in the context of their prior probabilities and matching scores (FIG. 5C). The PSMs reported by both methods receive generally higher matching scores and comparable prior probabilities. 825 out of 835 PSMs reported only by the database searching method received a global matching score below the threshold 0.0058 used for selecting de novo sequencing results. The remaining ten PSMs received prior probabilities less than 0.1 (on average, prior probability is 0.05), indicating they are less likely neoepitope peptides. On the other hand, among the top-ranked 27,476 PSMs reported only by the de novo sequencing algorithm, 23,857 have the prior probabilities above 0.1. The 18,905 unique peptides from these 27,476 top-ranked PSMs were further analyzed. When searching against the human protein database containing 21,006 sequences from Uniprot using Rapsearch2, 14,658 (77.53%) peptides have 50% or higher sequence similarity with some peptides from human proteins, while 7,737 (40.93%) peptides differ at at most two amino acids (i.e, a swap of two consecutive residues), including 1,910 (10.10%) identical peptides. Notably, although these identified peptides are more likely the true neoepitope peptides, some of the rest peptides may also be neoepitope peptides, e.g., those generated by novel gene splicing and fusion events, or PCPS.

Comparison with current de novo sequencing methods. The constrained de novo sequencing method was compared with the most recently developed de novo sequencing method uniNovo on the HLA-C peptidomic dataset. The parameters of uniNovo are chosen in consistence with the experimental settings: 1) the ion tolerance: 0.3 Da; 2) precursor ion tolerance: 100 ppm 3) fragmentation method: HCD; 4) no enzyme specificity is selected; 5) five peptide sequences per spectrum are reported; 6) minimum length of peptides: 9; and 7) minimum accuracy: 0.8. A total of 1,863 spectra are identified by uniNovo under these parameters. Most of the sequencing results are non-conclusive: only 3-6 (on average 3.1) amino acid residues were reported in these peptides, and the gaps between the residues were reported as mass intervals (e.g., a typical output of uniNovo is [406.2043]D[204.10266]QI). Because of the non-conclusive peptide sequences in uniNovo report, the resulting peptides were not compared to the results from the constrained de novo sequencing algorithms

Discussion

The constrained de novo sequencing method was designed specifically for characterizing neoepitope sequences from their MS/MS spectra acquired in immunopeptidomic experiments. The algorithm does not rely on a database of potential neoepitope peptides, and thus can identify peptides that are not contiguous subsequences of proteins in a database, including those resulting from novel insertion, deletion, splicing or gene fusion events, or those containing mutations (e.g., in tumor cells) or those generated by proteasome-catalyzed peptide splicing (PCPS). The dynamic programming algorithm adopted here allows for efficient searching in the entire space of peptide sequences within a range of desirable lengths (e.g., 9-12 residues). The results showed that, when peptides can be obtained by both methods, the peptide sequence reported by the de novo sequencing method often match with that from database searching, with at most one swap between two consecutive amino acid residues. Notably, unlike existing de novo sequencing algorithms (e.g., uniNovo) often reporting many putative sequence tags each with relatively low sequence coverage of target peptide, the constrained de novo sequencing method report one or a few complete peptide sequence with desirable length. As a result, it allows to search for the occurrence of peptide sequences in a protein database, even for those generated by PCPS (e.g., concatenated from two subpeptides in different proteins). It should be appreciated that one or more identified peptide epitopes may be selected and synthesized for cancer research and/or cancer immunotherapies.

The results on the testing dataset showed that many MS/MS spectra that were not identified by the database searching approach were identified as putative neoepitope peptides by the constrained de novo sequencing algorithm. This is probably due to the fact that the constrained de novo sequencing method benefits from the incorporation of PSSMs as prior probabilities, which prefers the peptides with high immunogenicities (i.e., likely to be presented by MHC-I). This is consistent with the typical experimental setting in immunopeptidomics, where peptides bound to a target MHC-I protein (e.g., HLA-C for the dataset used here) are enriched before the LC-MS/MS analyses. Hence, a majority of MS/MS spectra result from those peptides are anticipated and thus can be identified using the constrained de novo sequencing method. On the other hand, other peptides (not bound to the target MHC-I molecule) are not of interests in immunopeptidomics, and thus it is not a concern if the de novo sequencing method may not identify them.

Moreover, it should be appreciated that the preferences of MHC-I may be different in different patient because of the presence of many alleles of MHC-I encoding genes in human population. Therefore, specific PSSMs may be needed to be constructed for different MHC-I alleles so that appropriate PSSMs can be selected (based on HLA typing from the patient's genomic sequencing data) for neoepitope peptide analyses of an individual patient.

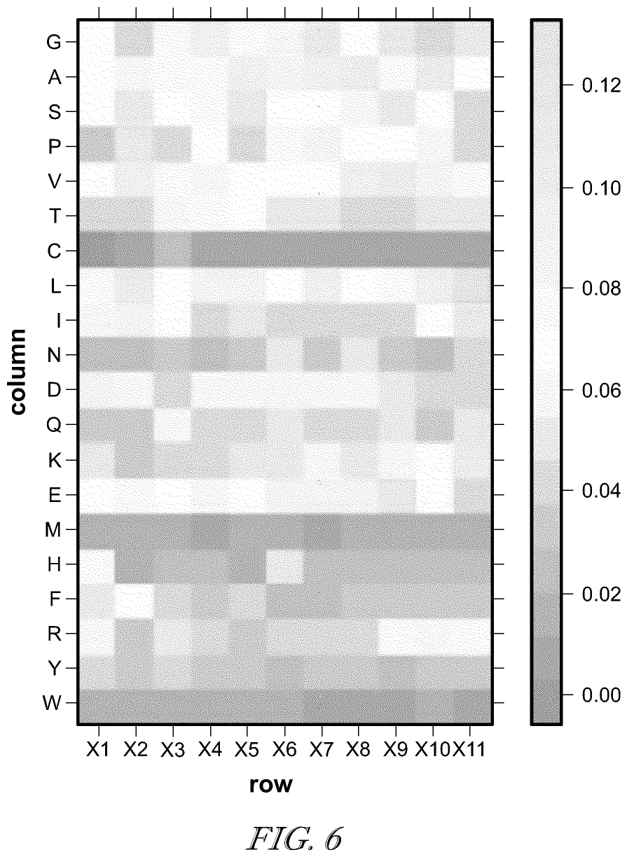

Even though the constrained de novo sequencing method of the present application was described and illustrated to characterize the peptides presented by MHC-I, this method can also be applied to sequencing of other types of neoepitope peptides. For example, the peptides presented by MHC-II that are important for CD4⁺ helper T-cell responses can also be characterized using a similar approach. The MHC-II presented peptides are typically longer in length and more variable (e.g., peptides with lengths of about 12 to about 18 residues), and thus more data are required to derive useful prior PSSM models. It should be appreciated that the average peptide length is about 15 residues long. An exemplary PSSM (shown as a frequency heatmap) derived from neoepitope peptides of 11 amino acid residues bound to HLA-C*0501 is shown in FIG. 6. Using the PSSM shown in FIG. 6, the constrained de novo algorithm was performed on MHC-II dataset obtained from Scholz E M et al., “Human Leukocyte Antigen (HLA)-DRB1*15:01 and HLA-DRB5*01:01 Present Complementary Peptide Repertoires,” FRONT IMMUNOL. 8:984 (2017). The dataset contains two replicated samples from HLA-DRB5*01:01. The constrained de novo algorithm identified 470 unique peptide epitopes from one replicate and 810 unique peptide epitopes from another replicate. It should be appreciated that one or more unique peptide epitopes may be selected and synthesized for antibiotic development and/or vaccine development.

There exist a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

### EXAMPLES

Example 1 includes a method comprising generating a database that includes peptide sequences within a predefined range of length of residues; assigning a prior probability to each of the peptide sequences in the database; obtaining mass spectra of a plurality of fragments of a target molecule produced by mass spectrometry; determining, for each mass spectrum, matching scores of peptide-spectrum matches between the mass spectra and the peptide sequences in the database as a function of prior probabilities of the peptide sequences and matching probabilities; and determining a subset of the peptide-spectrum matches that has a corresponding matching score higher than a threshold.

Example 2 includes the subject matter of Example 1, and wherein assigning the prior probability to each of the peptide sequences comprises assigning a prior probability to each of the peptide sequences in the database based on a positional specific scoring matric (PSSM) associated with each peptide length, wherein every position in the PSSM is determined based on an amino acid frequency.

Example 3 includes the subject matter of any of Examples 1 and 2, and wherein the prior probability of each peptide sequence is indicative of a probability of having a corresponding residue at each position.

Example 4 includes the subject matter of any of Examples 1-3, and wherein the matching probability is based on a probability of observing the mass spectrum from a target peptide sequence within the predefined range of length that maximizes a matching score based on a theoretical fragmentation of the target peptide sequence.

Example 5 includes the subject matter of any of Examples 1-4, and wherein the matching probability is a probability of observing an occurrence pattern of a set of fragment ions, including b-ion, y-ion, and neutral loss ions, derived from a fragmentation between fragmented peptides for each mass spectrum.

Example 6 includes the subject matter of any of Examples 1-5, and wherein assigning the prior probability to each of the peptide sequences in the database comprises assigning a higher prior probability to a peptide with a motif with higher immunogenicity.

Example 7 includes the subject matter of any of Examples 1-6, and further including outputting one or more peptide sequences in an order of the matching score corresponds to the peptide sequence.

Example 8 includes the subject matter of any of Examples 1-7, and wherein the database includes potential neoepitope peptides sequences.

Example 9 includes the subject matter of any of Examples 1-8, and wherein the predefined range of length of residues is 8-30 residues.

Example 10 includes the subject matter of any of Examples 1-9, and wherein the predefined range of length of residues is 9-12 residues.

Example 11 includes the subject matter of any of Examples 1-10, and wherein the predefined range of length of residues is 12-18 residues.

Example 12 includes the subject matter of any of Examples 1-11, and wherein obtaining mass spectra of a plurality of fragments comprises fragmenting a target molecule into a plurality of fragments by partial cleavage; performing mass spectrometry on the plurality of fragments to produce mass spectra of the fragments; and extracting peak information from the produced mass spectra.

Example 11 includes the subject matter of any of Examples 1-10, and further including pre-processing the mass spectra by removing at least one of peaks with an intensity of zero, a precursor peak, any converted mass greater than precursor mass, and isotopic masses of precursor masses.

Example 12 includes the subject matter of any of Examples 1-11, and wherein pre-processing the mass spectra further comprises logarithmically transforming intensities of all peaks.

Example 13 includes the subject matter of any of Examples 1-12, and further including selecting one or more peptide sequences from the subset of the peptide-spectrum matches; and synthesizing the one or more peptide sequences.

Example 14 includes one or more machine-readable storage media comprising a plurality of instructions stored thereon that, in response to being executed, cause a device to generate a database that includes peptide sequences within a predefined range of length of residues; assign a prior probability to each of the peptide sequences in the database; obtain mass spectra of a plurality of fragments of a target molecule produced by mass spectrometry; determine, for each mass spectrum, matching scores of peptide-spectrum matches between the mass spectra and the peptide sequences in the database as a function of prior probabilities of the peptide sequences and matching probabilities; and determine a subset of the peptide-spectrum matches that has a corresponding matching score higher than a threshold.

Example 15 includes the subject matter of Example 14, and wherein to assign the prior probability to each of the peptide sequences comprises to assign a prior probability to each of the peptide sequences in the database based on a positional specific scoring matric (PSSM) associated with each peptide length, wherein every position in the PSSM is determined based on an amino acid frequency.

Example 16 includes the subject matter of any of Examples 14 and 15, and wherein the prior probability of each peptide sequence is indicative of a probability of having a corresponding residue at each position.

Example 17 includes the subject matter of any of Examples 14-16, and wherein the matching probability is based on a probability of observing the mass spectrum from a target peptide sequence within the predefined range of length that maximizes a matching score based on a theoretical fragmentation of the target peptide sequence.

Example 18 includes the subject matter of any of Examples 14-17, and wherein the matching probability is a probability of observing an occurrence pattern of a set of fragment ions, including b-ion, y-ion, and neutral loss ions, derived from a fragmentation between fragmented peptides for each mass spectrum.

Example 19 includes the subject matter of any of Examples 14-18, and further including a plurality of instructions that in response to being executed cause the device to output one or more peptide sequences in an order of the matching score corresponds to the peptide sequence.

Example 20 includes the subject matter of any of Examples 14-19, and wherein the database includes potential neoepitope peptides sequences.

Example 21 includes the subject matter of any of Examples 14-20, and wherein the predefined range of length of residues is 8-30 residues.

Example 22 includes the subject matter of any of Examples 14-21, and wherein the predefined range of length of residues is 9-12 residues.

Example 23 includes the subject matter of any of Examples 14-22, and wherein the predefined range of length of residues is 12-18 residues.

Example 24 includes the subject matter of any of Examples 14-23, and wherein to obtain the mass spectra of a plurality of fragments comprises to fragment a target molecule into a plurality of fragments by partial cleavage; perform mass spectrometry on the plurality of fragments to produce mass spectra of the fragments; and extract peak information from the produced mass spectra.

Example 25 includes the subject matter of any of Examples 14-24, and further including a plurality of instructions that in response to being executed cause the device to pre-process the mass spectra by removing at least one of peaks with an intensity of zero, a precursor peak, any converted mass greater than precursor mass, and isotopic masses of precursor masses.

Example 26 includes the subject matter of any of Examples 14-25, and wherein to pre-process the mass spectra further comprises to logarithmically transform intensities of all peaks.

Example 27 includes the subject matter of any of Examples 14-26, and further including a plurality of instructions that in response to being executed cause the device to select one or more peptide sequences from the subset of the peptide-spectrum matches; and synthesize the one or more peptide sequences.

Example 28 includes a device comprising circuitry configured to generate a database that includes peptide sequences within a predefined range of length of residues; assign a prior probability to each of the peptide sequences in the database; obtain mass spectra of a plurality of fragments of a target molecule produced by mass spectrometry; determine, for each mass spectrum, matching scores of peptide-spectrum matches between the mass spectra and the peptide sequences in the database as a function of prior probabilities of the peptide sequences and matching probabilities; and determine a subset of the peptide-spectrum matches that has a corresponding matching score higher than a threshold.

Example 29 includes the subject matter of Example 28, and wherein to assign the prior probability to each of the peptide sequences comprises to assign a prior probability to each of the peptide sequences in the database based on a positional specific scoring matric (PSSM) associated with each peptide length, wherein every position in the PSSM is determined based on an amino acid frequency.

Example 30 includes the subject matter of any of Examples 28 and 29, and wherein the prior probability of each peptide sequence is indicative of a probability of having a corresponding residue at each position.

Example 31 includes the subject matter of any of Examples 28-30, and wherein the matching probability is based on a probability of observing the mass spectrum from a target peptide sequence within the predefined range of length that maximizes a matching score based on a theoretical fragmentation of the target peptide sequence.

Example 32 includes the subject matter of any of Examples 28-31, and wherein the matching probability is a probability of observing an occurrence pattern of a set of fragment ions, including b-ion, y-ion, and neutral loss ions, derived from a fragmentation between fragmented peptides for each mass spectrum.

Example 33 includes the subject matter of any of Examples 28-32, and wherein the circuitry is further configured to output one or more peptide sequences in an order of the matching score corresponds to the peptide sequence.

Example 34 includes the subject matter of any of Examples 28-33, and wherein the database includes potential neoepitope peptides sequences.

Example 35 includes the subject matter of any of Examples 28-34, and wherein the predefined range of length of residues is 8-30 residues.

Example 36 includes the subject matter of any of Examples 28-35, and wherein the predefined range of length of residues is 9-12 residues.

Example 37 includes the subject matter of any of Examples 28-36, and wherein the predefined range of length of residues is 12-18 residues.

Example 38 includes the subject matter of any of Examples 28-37, and wherein to obtain the mass spectra of a plurality of fragments comprises to fragment a target molecule into a plurality of fragments by partial cleavage; perform mass spectrometry on the plurality of fragments to produce mass spectra of the fragments; and extract peak information from the produced mass spectra.

Example 39 includes the subject matter of any of Examples 28-38, and wherein the circuitry is further configured to pre-process the mass spectra by removing at least one of peaks with an intensity of zero, a precursor peak, any converted mass greater than precursor mass, and isotopic masses of precursor masses.

Example 40 includes the subject matter of any of Examples 28-39, and wherein to pre-process the mass spectra further comprises to logarithmically transform intensities of all peaks.

Example 41 includes the subject matter of any of Examples 28-40, and wherein the circuitry is further configured to select one or more peptide sequences from the subset of the peptide-spectrum matches; and synthesize the one or more peptide sequences.

1.-41. (canceled)

42. A method comprising:
generating a database that includes peptide sequences within a predefined range of length of residues;
assigning a prior probability to each of the peptide sequences in the database;
obtaining mass spectra of a plurality of fragments of a target molecule produced by mass spectrometry;
determining, for each mass spectrum, matching scores of peptide-spectrum matches between the mass spectra and the peptide sequences in the database as a function of prior probabilities of the peptide sequences and matching probabilities; and
determining a subset of the peptide-spectrum matches that has a corresponding matching score higher than a threshold.

43. The method of claim 42, wherein assigning the prior probability to each of the peptide sequences comprises assigning a prior probability to each of the peptide sequences in the database based on a positional specific scoring matric (PSSM) associated with each peptide length, wherein every position in the PSSM is determined based on an amino acid frequency.

44. The method of claim 42, wherein the prior probability of each peptide sequence is indicative of a probability of having a corresponding residue at each position.

45. The method of claim 42, wherein the matching probability is based on a probability of observing the mass spectrum from a target peptide sequence within the predefined range of length that maximizes a matching score based on a theoretical fragmentation of the target peptide sequence.

46. The method of claim 45, wherein the matching probability is a probability of observing an occurrence pattern of a set of fragment ions, including b-ion, y-ion, and neutral loss ions, derived from a fragmentation between fragmented peptides for each mass spectrum.

47. The method of claim 42, wherein assigning the prior probability to each of the peptide sequences in the database comprises assigning a higher prior probability to a peptide with a motif with higher immunogenicity.

48. The method of claim 42, further comprising outputting one or more peptide sequences in an order of the matching score corresponds to the peptide sequence.

49. The method of claim 42, wherein the database includes potential neoepitope peptides sequences.

50. The method of claim 42, wherein the predefined range of length of residues is 8-30 residues.

51. The method of claim 42, wherein obtaining mass spectra of a plurality of fragments comprises:
fragmenting a target molecule into a plurality of fragments by partial cleavage;
performing mass spectrometry on the plurality of fragments to produce mass spectra of the fragments; and
extracting peak information from the produced mass spectra.

52. The method of claim 42, further comprising pre-processing the mass spectra by removing at least one of peaks with an intensity of zero, a precursor peak, any converted mass greater than precursor mass, and isotopic masses of precursor masses.

53. The method of claim 42, further comprising:
selecting one or more peptide sequences from the subset of the peptide-spectrum matches; and
synthesizing the one or more peptide sequences.

54. One or more machine-readable storage media comprising a plurality of instructions stored thereon that, in response to being executed, cause a device to:
generate a database that includes peptide sequences within a predefined range of length of residues;
assign a prior probability to each of the peptide sequences in the database;
obtain mass spectra of a plurality of fragments of a target molecule produced by mass spectrometry;
determine, for each mass spectrum, matching scores of peptide-spectrum matches between the mass spectra and the peptide sequences in the database as a function of prior probabilities of the peptide sequences and matching probabilities; and
determine a subset of the peptide-spectrum matches that has a corresponding matching score higher than a threshold.

55. The one or more machine-readable storage media of claim 54, wherein to assign the prior probability to each of the peptide sequences comprises to assign a prior probability to each of the peptide sequences in the database based on a positional specific scoring matric (PSSM) associated with each peptide length, wherein every position in the PSSM is determined based on an amino acid frequency.

56. The one or more machine-readable storage media of claim 54, wherein the prior probability of each peptide sequence is indicative of a probability of having a corresponding residue at each position.

57. The one or more machine-readable storage media of claim 54, wherein the matching probability is based on a probability of observing the mass spectrum from a target peptide sequence within the predefined range of length that maximizes a matching score based on a theoretical fragmentation of the target peptide sequence.

58. The one or more machine-readable storage media of claim 57, wherein the matching probability is a probability of observing an occurrence pattern of a set of fragment ions, including b-ion, y-ion, and neutral loss ions, derived from a fragmentation between fragmented peptides for each mass spectrum.

59. A device comprising:
circuitry configured to:
generate a database that includes peptide sequences within a predefined range of length of residues;
assign a prior probability to each of the peptide sequences in the database;
obtain mass spectra of a plurality of fragments of a target molecule produced by mass spectrometry;
determine, for each mass spectrum, matching scores of peptide-spectrum matches between the mass spectra and the peptide sequences in the database as a function of prior probabilities of the peptide sequences and matching probabilities; and
determine a subset of the peptide-spectrum matches that has a corresponding matching score higher than a threshold.

60. The device of claim 59, wherein to assign the prior probability to each of the peptide sequences comprises to assign a prior probability to each of the peptide sequences in the database based on a positional specific scoring matric (PSSM) associated with each peptide length, wherein every position in the PSSM is determined based on an amino acid frequency.

61. The device of claim 59, wherein to obtain the mass spectra of a plurality of fragments comprises to:
fragment a target molecule into a plurality of fragments by partial cleavage;
perform mass spectrometry on the plurality of fragments to produce mass spectra of the fragments; and
extract peak information from the produced mass spectra.
